(19) United States
(12) Patent Application Publication
(10) Pub. No.: US 2009/0068125 A1
(43) Pub. Date: Mar. 12, 2009

(54) TREATMENT FOR MUCOSITIS

(75) Inventors: Richard Mark Edwards, Bucks (GB); John Martin Clements, Oxon (GB)

Correspondence Address: SEED INTELLECTUAL PROPERTY LAW GROUP PLLC, 701 FIFTH AVE, SUITE 5400, SEATTLE, WA, 98104, US

(73) Assignee: HEPTAGEN LTD, Alesbury (GB)

(21) Appl. No.: 11/814,796

(22) PCT Filed: Jan. 25, 2006
(86) PCT No.: PCT/GB2006/000241

§ 371 Date: Oct. 27, 2008

(30) Foreign Application Priority Data
Jan. 25, 2005 (GB) 0501476.6

(51) Int. Cl. A61K 8/60; C07H 19/167; C07H 19/20; C07H 19/16; A61K 31/52; A61K 31/675

(52) U.S. Cl. 424/57; 536/27.6; 536/26.7; 536/27.8; 536/27.2; 514/46; 514/47; 514/45; 424/49

(57) ABSTRACT

This invention relates to a medicament for protecting human mucosal cells from chemotherapy or radiotherapy induced cell death using formulations comprising adenosine or adenosine analogues as a reversible inhibitor of epithelial cell proliferation. The invention also relates to formulations containing adenosine for the prevention of or reduction in mucositis symptoms.

The present invention relates to a method for the treatment of mucositis and to compositions useful for this purpose. In particular, the invention relates to the use of adenosine or adenosine analogues in the treatment of mucositis.

Oral mucositis is inflammation of the mucosa in the mouth. It occurs as a common side effect of chemotherapy and radiation treatment. Because cytotoxic therapy targets dividing cells, it is the immune system and epithelial layers that are most vulnerable, explaining why GI tract disturbance, immunosuppression and alopecia are the most obvious and treatment limiting side-effects. In the mouth, killing epithelial progenitor cells inhibits the ability of the mucosal layer to repair microlesions, leading to ulceration exacerbated by infection.

According to the National Cancer Institute, oral mucositis occurs in almost all patients receiving radiation for head and neck malignancies, in more than 75 percent of bone marrow transplant recipients, and in nearly 40 percent of patients receiving chemotherapy. Patients with reduced immune response, such as HIV/AIDS may also face this condition. Until recently, no truly effective treatment for mucositis was available. Approximately 400,000 patients in the United States experience mucositis and the market potential is estimated at $300 million to $500 million. An important point is that mucositis often represents a treatment limiting side effect, and there is therefore an indirect effect on the overall efficacy of cancer treatment.

Available treatment strategies fall into two main categories:

Preventative, taken before and during radiotherapy or chemotherapy aim to protect mucosal epithelial cells, by taking them out of cell-cycle or reducing the damaging effects of free-radicals. For example, one approach is to suck ice cubes during bolus 5-FU treatment. This suppresses cellular activity, rendering the mucosal progenitor cells less susceptible to damage. Vitamin E and beta-carotene have been used as cytoprotectants.

Palliative treatments reduce the pain and discomfort that is characteristic of the condition. They generally act to keep the mucosal surface moist and may have anaesthetic properties. Anti-microbial agents act to reduce the effect of infection. Some agents actively coat the mucosal surface, protecting the ulcer and reducing discomfort.

Overall, there are a large number of empirical approaches to the management of mucositis, but few of these are supported by compelling objective evidence based on clinical trials.

Until recently, there were no treatments that actively promote resolution of mucositis, though there is objective evidence that G-CSF or GM-CSF injected subcutaneously may help reduce symptoms and accelerate healing. This effect is probably indirect, and mediated by the effect of these agents in stimulating the recovery from neutropenia and reducing infection.

The newer biological agents are, however, showing efficacy in the clinic, with Amgen's Palifermin (keratinocyte growth factor) receiving FDA approval in December 2004. These agents will be expensive, however, and there is concern over the administration of growth factors, given their potential for stimulating the growth of tumours and the mutagenic nature of most cancer treatment.

There is therefore a clear and continuing need for better treatments for mucositis, especially those having a different mode of action to growth factors.

Adenosine and its analogues can be used as a cryoprotectant before each cancer treatment to remove oral epithelial cells from cycle and reduce their sensitivity to cytotoxicity.

Normally, cells trying to divide after receiving DNA damage would undergo apoptosis as a defence against the emergence of pre-cancerous and cancerous clones of cells. Under cytotoxic treatments using drugs or radiotherapy, however, this defence mechanism leads to the ablation of normal cell populations. If it were possible to prevent the cells from dividing until DNA repair mechanisms had acted, it would be possible to reduce the cytotoxic side-effects of chemotherapy and reduce damage to the mucosal layer. This would reduce the symptoms and/or enhance the rate of recovery.

The invention is based on the observation that adenosine is a potent and reversible inhibitor of epithelial cell proliferation (GB0409129.4; GB0413380.7; Cook et al. J. Inv. Derm. 104; 976-981) and has properties which make it ideal for the amelioration of mucositis.

The invention relates to a method for the treatment of mucositis, the method comprising administering to a patient in need of such treatment an effective amount of an adenosine receptor agonist.

In a first aspect of the invention, therefore, there is provided the use of an adenosine receptor agonist in the preparation of an agent for the treatment or prevention of mucositis.

The use of an adenosine receptor agonist in the treatment or prevention of mucositis has the following advantages.

1) The lack of keratinisation and consequently greater permeability of the buccal and sub-lingual mucosa allow the ready local delivery of adneosine.

2) The reversible inhibition of keratinocytes and epithelial stem cells provide a means to remove these vulnerable cells from the mitotic compartment during pulsed chemotherapy or radiotherapy.

3) The very short half-life of adenosine in the circulation (<10s) prevents any systemic effects, ensuring that cyto-protection was short-lived and local, and preventing any cardiac effects resulting from systemic exposure to the drug.

In the context of the present invention, the term “adenosine receptor agonist” refers to a compound which is capable of stimulating a human adenosine receptor when expressed in Chinese hamster ovary (CHO) cells.

Expression of a human adenosine receptor in Chinese hamster ovary cells can be achieved by standard techniques known to those of skill in the art. Examples of suitable protocols can be found in Iredale et al., Br. J. Pharmacol. 1994, 111(4), p 1252-1256, Kull et al., Biochem. Pharmacol. 1999, 57(1), p 65-75 and Salvatore et al., Proc. Natl. Acad Sci USA, 1993, 90(21), p 10365-10369.

Typically, an adenosine receptor agonist is a compound which has a maximal in vitro activity in stimulating human adenosine receptors expressed in CHO cells which is at least 25%, preferably at least 50%, more preferably at least 75%, of the maximal activity achieved with adenosine under identical assay conditions. The said adenosine receptor can be an A1, A2A, A2B or A3 receptor.

The preferred adenosine agonist is adenosine itself. Other useful compounds include ATP, ADP and AMP, inosine and other purines and purine nucleotides and, where appropriate, pharmaceutically acceptable salts of any of these.

Adenosine is a natural compound, generally recognised as safe in the diet. Although it is a prescription only medicine as a systemic agent used to treat cardiac arrhythmias, regulatory authorities have accepted that oral dosing with adenosine in vitamin supplements is exceedingly unlikely to give rise to systemic effects.

In order for the invention to be effective, it is necessary for the adenosine receptor agonist to be administered to the oral mucosa and it will therefore be formulated in a manner which makes this possible.

In a further aspect of the invention, there is provided a pharmaceutical composition comprising an adenosine receptor agonist in a liquid or semi-solid base.

The formulation of liquid or semi solid bases is well known to one skilled in the art of pharmaceutics.

The adenosine agonist may be either in solution or suspension, or a mixture of the two in the liquid or semi-solid base.

A liquid pharmaceutical composition may be a mouthwash and will preferably have a pH of 3.5 to 8. A pH of 4 to 6.5 is most preferable as a preparation having a pH of less than about 4 would be likely to cause a stinging sensation, while preparations having a pH greater than about 6.5 are often unpleasant to use.

Appropriate buffer systems include citrate, acetate, tromethamine and benzoate systems. However, any buffer system commonly used for preparing medicinal compositions would be appropriate.

While the vehicle used generally is primarily water, other solvents may be present. For example, solvents such as alcohols, glycols (propylene glycol, polyethylene glycol or polypropylene glycol are examples), glycerin, and the like may be used to solubilize the active agents.

The compositions may also contain surfactants, for example anionic, nonionic, amphoteric and cationic surfactants, many of which are known in the art as appropriate ingredients for mouthwashes.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyallcyl, and hydroxyalkyl alkyl celluloses, acrylates, poloxamer, alginates, pectins, guar gum, polyvinylpyrolidone, and gellan gums. High viscosity formulations may cause nausea in chemotherapy and radiation patients and are therefore not preferred. Gellan gums are preferred as viscosity modifying agents since aqueous solutions containing certain gellan gums may be prepared so that they will experience an increase in viscosity upon contact with electrolytes. Saliva contains electrolytes that will interact with such a gellan containing solution so as to increase their viscosity.

Lotions and light creams maybe formulated by incorporation of a range of emollient oils including paraffin and other hydrocarbon based oils, vegetable oils and modified vegetable oils, silicones and the like as described in the general literature for example “Dermatological Formulation” (B W Barry, Marcel Dekker, 1983. ISBN 0-824-1729-5).

Ointment-type bases, especially those including polymers as mentioned previously which increase retention may also be used as bases for the current invention.

Alternatively, the adenosine receptor agonist may be provided as a solid formulation which dissolves in the mouth.

Therefore, in a further aspect of the invention, there is provided a solid pharmaceutical composition which is adapted to dissolve in the mouth and which comprises an adenosine agonist.

The solid formulation may take the form of a powder, tablet, troche, pastille or lozenge. Especially in the case of pastilles and lozenges it may be advantageous for the active to be in a solid solution or molecular dispersion within the formulation.

In order to improve the patient acceptability, it is desirable to add an appropriate colouring and/or flavouring material to both the liquid and solid compositions of the present invention. Any pharmaceutically acceptable colouring or flavouring material may be used.

For example, flavorings used in the mouthrinse art such as peppermint, citrus flavorings, berry flavorings, vanilla, cinnamon, and sweeteners, either natural or artificial, may be used. As an additional benefit, flavourings that are known to increase salivary electrolyte concentrations may be added to increase the magnitude of the viscosity change obtained with gellan gums. The increased viscosity will promote retention of the solutions in the oral cavity and provide greater effectiveness due to increased contact time with the affected tissues.

Antimicrobial preservatives may be present in the liquid and solid formulations in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

The liquid or solid compositions may also comprise a penetration enhancer to improve delivery to the basal layers of the epithelia.

Suitable penetration enhancers include: 23-lauryl ether, benzalkonium chloride, cetylpyridinium chloride, cyclodextrin, lauric acid/propylene glycol, lysophosphatidylcholine (LPC), menthol, phosphatidylcholine, sodium lauryl sulfate.

Preferably the penetration enhancer will possess additional useful properties. LPC is preferred because it also has keratinocyte inhibition properties. Possible enhancers are well known to persons skilled in the art; see for example “Buccal mucosa as a route for systemic drug delivery: a review. Shojaei A H; J Pharm Pharm Sci. 1998; 1:15-30”.

A further optional ingredient of the compositions is an inhibitor of adenosine deaminase, which potentiates the effect of the adenosine receptor agonist by inhibiting its degradation by adenosine deaminase in the plasma.

A suitable adenosine deaminase inhibitor is inosine, which reduces breakdown by product inhibition (A product inhibition study on adenosine deaminase by spectroscopy and calorimetry. Saboury A A; J Biochem Mol Biol. 2002; 35:302-5). Other inhibitors include natural compounds like caffeine, and synthetic analogues such as the PDE2 inhibitor EHNA hydrochloride (erythro-9-(2-Hydroxy-3-nonyl)adenine hydrochloride) (Bessodes et al, Biochem. Pharmacol. 31; 879) or pentostatin (2-deoxycoformycin).

The adenosine receptor agonist may also be provided as part of a composition which can be dissolved or suspended in a liquid vehicle to form a liquid pharmaceutical composition as described above.

In a further aspect of the invention, therefore, there is provided a solid composition comprising adenosine or an adenosine agonist which is can be rapidly dissolved or dispersed in a liquid vehicle to form a liquid pharmaceutical composition as described previously.

The solid composition preferably takes the form of a powder or tablet which contains an adenosine receptor agonist which has been treated by a process selected from freeze-drying, spray-drying, particle-size reduction.

The invention will now be described in greater detail with reference to the Example and to the drawing in which:

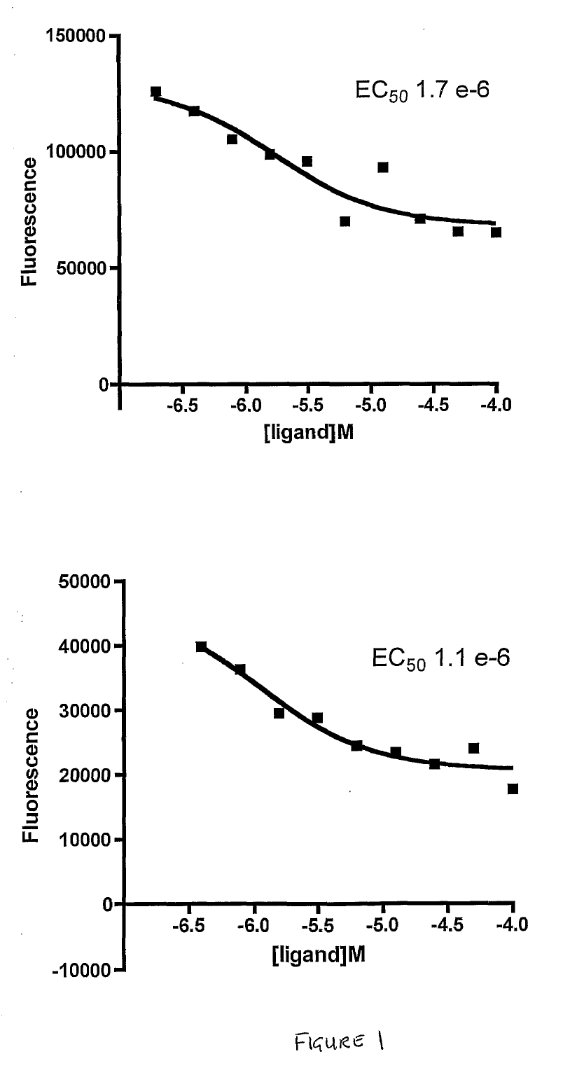

FIG. 1 is a set of two plots showing the inhibition of keratinocyte proliferation by adenosine. It can be seen that keratinocyte proliferation declines as the concentration of adenosine increases.

### EXAMPLE 1

### Adenosine is a Potent Inhibitor of Keratinocyte Proliferation

Cryopreserved human epidermal keratinocytes (HEK) isolated from skin were obtained from TCS Cellworks, Botolph Claydon, Buckingham, MK18 2LR, UK. Cells were maintained in EpiLife, a defined basal medium designed for human keratinocytes supplemented with selected hormones and growth factors (TCS Cellworks). Cells were maintained for a maximum of 15 population doublings to ensure the cultures do not terminally differentiate.

Proliferating cultures were trypsinised, harvested, treated with a trypsin inhibitor and resuspended in growth medium. The viable cells were counted then replated into 24 or 96 well cell culture plates at a density of approximately 2,500 cells/cm2. Cells were incubated overnight at 37° C. at 5% CO2 to allow recovery, the spent medium aspirated from the wells and replaced with fresh growth medium in the presence or absence of adenosine.

Adenosine was obtained from Sigma-Aldrich and made up as a 10 mM stock solution in DMSO. Serial dilutions were made in the growth medium and added to the cells to give final concentrations ranging from 0.1 to 100 microM, and the cells returned to the incubator for 2 days.

AlamarBlue 2% v/v was added to the wells and reduction measured fluorometrically using a Molecular Dynamics BioLumin 9600 microtitre plate reader using excitation wavelength at 530 nM and absorbance wavelength at 590 nM.

The results of two typical experiments are shown in FIG. 1. Adenosine gave a dose-dependent inhibition of keratinocyte proliferation with EC50 values of 1.7 and 1.1 micromolar.

Control experiments in which compounds were not added were carried out to examine the normal time-scale of proliferation. The cells typically grew to give an overall two to three-fold in cell numbers after 2-5 days. We would therefore expect to see two patterns of growth inhibition. Compounds that only inhibit cell-proliferation would be expected to cause a reduction of ˜50% in fluorescence. Compounds that caused a greater than 50% reduction are likely to be causing cell-death.

The pattern of inhibition seen with adenosine is consistent with its role as a reversible inhibitor of adenosine proliferation, indicating its suitability as a cytoprotectant.

1. The use of an adenosine receptor agonist in the preparation of an agent for the treatment or prevention of mucositis.

2. The use as claimed in claim 1 wherein the adenosine receptor agonist is adenosine, adenosine triphosphate, adenosine diphosphate, adenosine monophosphate, inosine a derivative or a purine nucleotide or, where appropriate, a pharmaceutically acceptable salt of any of these.

3. A pharmaceutical composition comprising an adenosine receptor agonist in a liquid or semi-solid base.

4. A pharmaceutical composition as claimed in claim 3, wherein the liquid or semi-solid base is aqueous.

5. A pharmaceutical composition as claimed in claim 3 or claim 4 which is a mouthwash.

6. A pharmaceutical composition as claimed in any one of claims 3 to 5 which has a pH of 3.5 to 8.

7. A pharmaceutical composition as claimed in claim 6, which has a pH of 4 to 6.5

8. A pharmaceutical composition as claimed in any one of claims 3 to 7, which is buffered using a buffer system selected from citrate, acetate, tromethamine and benzoate systems.

9. A pharmaceutical composition as claimed in any one of claims 3 to 8 which, in addition to the liquid or semi-solid base, contains a further solvent chosen from alcohols, glycols and glycerin.

10. A pharmaceutical composition as claimed in any one of claims 3 to 9, further comprising a surfactant.

11. A pharmaceutical composition as claimed in any one of claims 3 to 10, further comprising a viscosity increasing agent.

12. A solid pharmaceutical composition which is adapted to dissolve in the mouth and which comprises an adenosine agonist together with a suitable excipient.

13. A solid pharmaceutical composition as claimed in claim 12 which is a powder, tablet, troche, pastille or lozenge.

14. A pharmaceutical composition as claimed in any one of claims 3 to 13, further comprising a colouring and/or flavouring material.

15. A pharmaceutical composition as claimed in any one of claims 3 to 14, further comprising an antimicrobial preservative.

16. A pharmaceutical composition as claimed in any one of claims 3 to 15, further comprising a penetration enhancer to improve delivery to the basal layers of the epithelia.

17. A pharmaceutical composition as claimed in claim 16, wherein the penetration enhancer is 23-lauryl ether, benzalkonium chloride, cetylpyridinium chloride, cyclodextrin, lauric acid/propylene glycol, lysophosphatidylcholine (LPC), menthol, phosphatidylcholine or sodium lauryl sulfate.

18. A pharmaceutical composition as claimed in claim 17, wherein the penetration enhancer is lysophosphatidylcholine.

19. A pharmaceutical composition as claimed in any one of claims 3 to 18, further comprising an inhibitor of adenosine deaminase such as inosine.

20. A solid composition comprising adenosine or an adenosine agonist which is adapted to be rapidly dissolved or dispersed in a liquid vehicle to form a pharmaceutical composition as claimed in any one of claims 3 to 11.

21. A composition as claimed in any one of claims 3 to 20, wherein the adenosine receptor agonist is adenosine, adenosine triphosphate, adenosine diphosphate, adenosine monophosphate, inosine a purine derivative or a purine nucleotide or, where appropriate, a pharmaceutically acceptable salt of any of these.
